(19) United States
(12) Patent Application Publication
(10) Pub. No.: US 2020/0000597 A1
(43) Pub. Date: Jan. 2, 2020

(54) TRIAL FEMORAL HEAD PROSTHESIS

(71) Applicant: DEPUY IRELAND UNLIMITED COMPANY, COUNTY CORK (IE)

(72) Inventors: CONOR LOWRY, LEEDS (GB); DUNCAN YOUNG, MELBOURN (GB)

(21) Appl. No.: 16/486,519

(22) PCT Filed: Jan. 24, 2018
(86) PCT No.: PCT/EP2018/051645

§ 371 Date: Aug. 16, 2019

(30) Foreign Application Priority Data
Feb. 17, 2017 (GB) 1702610.5

(51) Int. Cl. A61F 2/36; A61F 2/46

(52) U.S. Cl. CPC A61F 2/3609; A61F 2002/30242; A61F 2/4684

(57) ABSTRACT

A trial femoral head for use in a trial fitting of a hip prosthesis, the head having an outer surface and a plurality of cavities configured to mate with a male connection member of a femoral stem component, each cavity having an opening located in the outer surface of the head, each opening being of a different size such that when the male connection member of the femoral stem component is mated with a first cavity it is received at a first depth, and when it is mated with a second cavity it is received at a second depth.

### FIELD OF THE INVENTION

The present invention relates to trial femoral heads for use with femoral stem components, kits including the trial femoral heads and methods of use thereof.

### BACKGROUND TO THE INVENTION

Hip arthroplasty is often used to restore function to a diseased or injured hip joint. In total hip arthroplasty, surfaces of the femur and pelvis are prepared, such as by cutting and/or reaming, and replaced with substitute implants. Hip arthroplasties are generally based on a modular construction. The implants often include a femoral stem component, a femoral head component, and an acetabular prosthesis. The acetabular prosthesis conventionally consists of two components: an acetabular cup and a liner. This modularity enables the surgeon to optimize reconstruction of the original anatomy. Mechanically, the goals of hip arthroplasty include, creating a stable articulation with optimised range of motion, restoring biomechanics for muscular efficiency and equalised limb lengths.

During a hip arthroplasty procedure it is often desired to use a provisional or trial femoral head component prior to the fitting of the definitive prosthetic femoral head component. In the trialing procedure, the trial head is temporarily secured to the femoral stem component. The femoral head component is then inserted into an acetabular component. The acetabular component may be the patient's natural acetabulum or an implanted acetabular cup. A trial reduction of the joint is then performed. This enables the femoral head component to be realistically evaluated for fit, joint tension, range of motion, and the jumping distance (i.e., the degree of lateral translation of the femoral head center required before dislocation occurs). The trial femoral head may be replaced with other trial heads as necessary until the desired neck length is reached. The trial femoral head is then removed and replaced with a definitive prosthetic femoral head which corresponds to the selected trial head.

Femoral head components are offered in multiple neck lengths. This is often also referred to as the head offset. Each manufacturer tends to uses its own notation to identify the various offered neck lengths. For example, DePuy Synthes offers, hip systems that include trial and definitive femoral head components that have 3.5 mm increments (e.g. 1.5 mm, 5 mm, 8.5 mm, 12 mm and 15.5 mm) in neck length.

An instrument kit also conventionally includes a range of trial femoral head components that differ in head diameter (e.g., 32 mm, 36 mm, 40 mm, etc. . . . ). As each size of trial head component is also offered with a different neck length (e.g., 1.5 mm, 5 mm, 8.5 mm, etc. . . . ), multiple Stock Keeping Units (SKUs) have to be created. Not only is this costly, but the required inventory takes up space within the instrument kit, and adds weight to the instrument kits.

In addition, when a surgeon wants to switch between different neck lengths, s/he has to remove the trial femoral head, hand it to an assistant and request a trial femoral head component that will provide a different neck length. An assistant then has to select the correct component from within the instrument kit, and pass it to the surgeon for mating with the femoral stem component. This is time consuming. This process may be repeated numerous times, for instance, a surgeon may trial a 5 mm neck length, then trial a 8.5 mm neck length, and then revert back to the 5 mm neck length, before deciding on the neck length of the definitive femoral head component. This process requires the surgeon to twist in a manner which is not ergonomic. It also requires the surgeon and the assistant to handle spherical components that are coated in blood and hence slippery, multiple times. This increases the risk that the component will be dropped or become lost within the wound site.

There is therefore a need for an improved trial femoral head component that reduces the inventory required within an instrument kit. There also a need for a trial femoral head component that improves the surgical process, for example temporally and ergonomically.

### SUMMARY OF THE INVENTION

According to an aspect of the invention there is provided a trial femoral head for use in a trial fitting of a hip prosthesis, the head having an outer surface and a plurality of cavities configured to mate with a male connection member of a femoral stem component, each cavity having an opening located in the outer surface of the head, each opening being of a different size such that when the male connection member of the femoral stem component is mated with a first cavity it is received at a first depth, and when it is mated with a second cavity it is received at a second depth.

According to a further aspect of the invention, there is provided a kit comprising first and second trial femoral heads for use in a trial fitting of a hip prosthesis, each head having an outer surface and a plurality of cavities configured to mate with a male connection member of a femoral stem component, each cavity having an opening located in the outer surface of each head, each opening being of a different size such that when the male connection member of the stem component is mated with a first cavity it is received at a first depth, and when it is mated with a second cavity it is received second depth, and in which the outer surface of the first trial femoral head has a diameter that is different from a diameter of the outer surface of the second trial femoral head.

According to a further aspect of the invention there is provided a hip prosthesis trial system comprising:

at least one trial femoral head for use in a trial fitting of a hip prosthesis, the head having an outer surface and a plurality of cavities configured to mate with a male connection member of a femoral stem component, each cavity having an opening located in the outer surface of the head, each opening being of a different size such that when the male connection member of the femoral stem component is mated with a first cavity it is received at a first depth, and when it is mated with a second cavity it is received at a second depth; and

at least one femoral stem component having a male connection member configured to mate with each of the plurality of cavities within the trial femoral head.

According to a still further aspect of the invention there is provided a method of trialing, comprising the steps of:

using a trial femoral head having an outer surface and a plurality of cavities configured to mate with a male connection member of a stem component, each cavity having an opening located in the outer surface of the head, each opening being of a different size such that when the male connection member of the stem component is mated with a first cavity it is received at a first depth, and when it is mated with a second cavity it is received at a second depth,

connecting the trial femoral head onto an implanted femoral stem component by inserting the male connection member into a first cavity of the trial femoral head;

inserting the trial femoral head into an acetabulum of a patient or into an acetabular cup located in the acetabulum; and

performing a trial reduction to form a trial joint construct.

The method may also comprise the steps of:

(i) conducting a trial range of motion assessment; and/or

(ii) evaluating stability of the trial joint construct, and/or

(iii) evaluating leg length provided by the trial joint construct.

Optionally, the method may also the surgeon trialing a different neck length. This involves the additional steps of removing the male connection member of the stem component from the first cavity and inserting the male connection member into a second cavity of the trial femoral head. The trial head is then inserted into an acetabular cup or into an acetabular cup located in the acetabulum. A trial reduction is then performed to form a trial joint construct.

The method may also comprises the steps of:

conducting a trial range of motion assessment; and/or

(ii) evaluating stability of the trial joint construct, and/or

(iii) evaluating leg length provided by the trial joint construct.

In some constructions of the trial femoral head, each cavity comprises a tapered surface to engage with a tapered male connection member. The male tapered connection member is often referred to as a trunnion. The tapered connection between the cavity of the femoral head and the tapered male connection member of the femoral stem may be in the form of a self-locking taper (Morse). The Morse taper may be a 12/14 taper, although other tapered connections are envisaged.

Optionally, a taper angle of the tapered surface of each cavity is the same.

Each cavity has a male connection member insertion axis extending inwardly from the opening. In some constructions of the trial femoral head, the male connection member insertion axis of the first cavity is co-axial with the male connection member insertion axis of the second cavity. In other constructions of the trial femoral head the male connection member insertion axis of the first cavity is oriented at a non-zero angle relative to the male connection member insertion axis of the second cavity. Optionally, the male connection member insertion axis of the first cavity is orthogonal to the male connection member insertion axis of the second cavity.

Conventional trial femoral heads include at least one suture hole. The formation of this suture hole increases the complexity of the manufacturing process.

Advantageously therefore, in some constructions of the trial femoral head, the first and second cavities are interconnected as a result of the tapered surface of the first cavity connecting with the tapered surface of the second cavity. This interconnection enables a suture to be passed through both of the cavities for retention of the head within the surgical site during surgery. In some other constructions, each of the first and second cavities includes an end wall located opposite to the opening. The end wall of the first and second cavity may be provided with a suture hole. A suture channel may extend between the suture hole of the first and second cavities. The combination of a suture hole/suture channel provides an interconnection between the two cavities through which a surgeon may pass a suture.

The trial femoral head may include a third cavity which has a male connection member insertion axis that is angled at a non-zero angle, for example orthogonal, relative to the male connection member insertion axis of at least one of the first and second cavities.

In some constructions of the trial femoral head the third cavity comprises a tapered surface to engage with a tapered male connection member, and this tapered surface may connect with the tapered surface at least one of the first and second cavities. This provides an interconnection between at least two of the cavities, thereby enabling a suture to be used for retention of the trial femoral head within the surgical site, as described above. In some other constructions, the third cavity includes an end wall located opposite to the opening. The end wall of the third cavity may be provided with a suture hole. A suture channel may extend between the suture hole of the third cavity and a suture hole provided in an end wall of the first and/or second cavity. The combination of a suture hole/suture channel provides an interconnection between at least two of the cavities, through which a suture may be threaded.

In some other constructions of the trial femoral head, the head further includes a fourth cavity which has a male connection member insertion axis that is at a non-zero angle, for example orthogonal, relative to the male connection member insertion axis of at least one of the first, second or third cavities.

It is also envisaged that the male connection member insertion axis of the fourth cavity may be co-axial with the male connection member insertion axis of the third cavity.

In some constructions of the trial femoral head the fourth cavity comprises a tapered surface to engage with a tapered male connection member, and this tapered surface may connect with the tapered surface at least one of the first, second and third cavities. This provides an interconnection between at least two of the cavities, thereby facilitating a suture to be used for retention of the trial femoral head within the surgical site, as described above. The end wall of the fourth cavity may be provided with a suture hole. A suture channel may extend between the suture hole of the fourth cavity and a suture hole provided in an end wall of the first, second and/or third cavity. The combination of a suture hole/suture channel provides an interconnection between at least two of the cavities, through which a suture may be threaded.

The provision of two cavities on the trial femoral head instead of the conventional one cavity reduces the inventory required within the instrument kit by 50%. Taking as an example an instrument kit that includes conventional (i.e., one cavity) trial femoral heads offered in a range of head diameters (i.e., 32 mm, 3 6 mm, 38 mm and 40 mm) each head diameter also being offered with a different neck length (i.e., 1.5 mm, 5 mm, 8.5 mm and 12 mm), the kit would have an SKU of 16 trial femoral heads. In comparison, according to the present invention, when the femoral head is provided with two cavities, the SKU can be reduced to 8. The provision of four cavities on the trial femoral heads would reduce the SKU further to 4 trial femoral heads. Not only does this reduce the cost of the instrument kit, but also the space taken up in the kits by the trial femoral heads and the weight of the kits.

Each cavity is advantageously provided with indicia to indicate to the surgeon the neck length that the connection of that cavity of the trial femoral head to the male connection member of the stem component will provide (e.g., 1.5 mm, 5 mm, 8.5 mm, etc. . . . ). For example, each cavity may have a different colour of indicia associated with it that indicates the resulting neck length. In some constructions a ring of a coloured label may be provided around the perimeter of the opening.

The provision of more than one cavity on the trial femoral head also improves the handling of the trial component. For example, the cavities that are unused during trialing can function as extra grip features, or can enable the trial femoral head to be more readily gripped by forceps.

Advantageously, the provision of more than one cavity on the trial femoral head provides an ergonomic benefit by minimising the need for the surgeon to alter his/her stance (e.g., by twisting each time a trial head is passed back and forth between the surgeon and an assistant) during the surgical procedure. This also provides an improvement in the time efficiency of the procedure, for example, the surgeon does not need to wait until an assistant locates the requested trial femoral head within the instrument kit and passes it to him/her. Instead the surgeon simply rotates the trial femoral head and trials the joint assembly using a different cavity.

It is envisaged that a trial femoral head may have 2 cavities, or 3 cavities or 4 cavities or more than 4 cavities, for example 5 or 6 cavities. However, it is particularly advantageous that the outer surface of the trial femoral head is generally spherical and has a smooth surface, thus resembling a conventional trial femoral head. This will provide a surgeon with confidence that the modified design will function biomechanically in a similar manner to a conventional trial femoral head during trialing.

The surgeon may utilize only one of the cavities on the trial femoral head. This might be the case if s/he decides that the neck length achieved by the connection of the trial head on the stem component will provide the appropriate final neck length. However, the surgeon may utilize at least another cavity provided within the femoral head during the trialing procedure.

For example, the surgeon may undertake a first trial procedure by inserting a male connection member of the femoral stem component inserted into a first cavity to provide a first neck length. If the surgeon is uncertain as to whether the optimal neck length is provided, s/he may then remove the trial femoral head, rotate it and insert the male connection member of the femoral stem component into a second cavity. In this configuration the male connection member is received at a different depth within the second cavity to when it is received within the first cavity. This enables the surgeon to undertake a second trial procedure at a second neck length.

Optionally, the surgeon may remove the trial femoral head, rotate it and insert the male connection member of the femoral stem component back into a first cavity, and re-trial.

Optionally, the surgeon may remove the trial femoral head, rotate it and insert the male connection member of the femoral stem component into at least a third cavity, with the male connection member being received at a different depth when it is received within the third cavity to when it is received within the first and second cavities. This enables a surgeon to undertake at least a third trial procedure at a third neck length.

The formation of cavities within a generally spherical surface will result in each opening of each cavity being substantially flat when viewed in a direction substantially orthogonal to the male connection member insertion axis. The flat areas created by the openings may function as anti-roll features.

It is also envisaged that the trial femoral head may also function as an insertion tool for an acetabular cup liner.

The trial femoral head of the present invention may be produced by machining or injection moulding.

### DETAILED DESCRIPTION OF THE DRAWINGS

The invention will now be described, by way of example only, with reference to the following drawings in which:

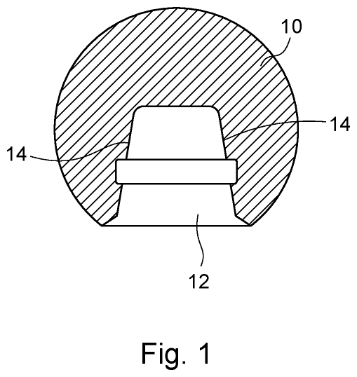

FIG. 1 is schematic of a conventional trial femoral head.

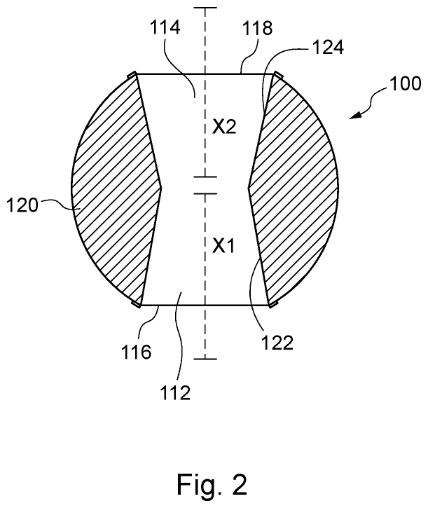

FIG. 2 is a schematic of a first construction of the trial femoral head.

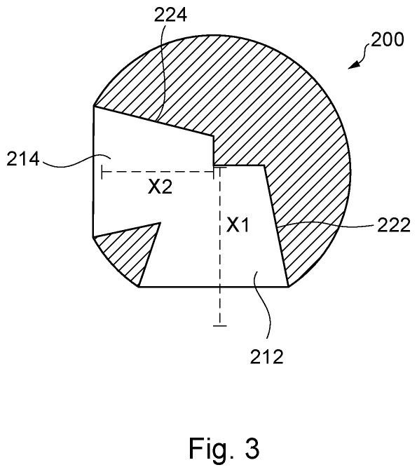

FIG. 3 is a schematic of a second construction of the trial femoral head.

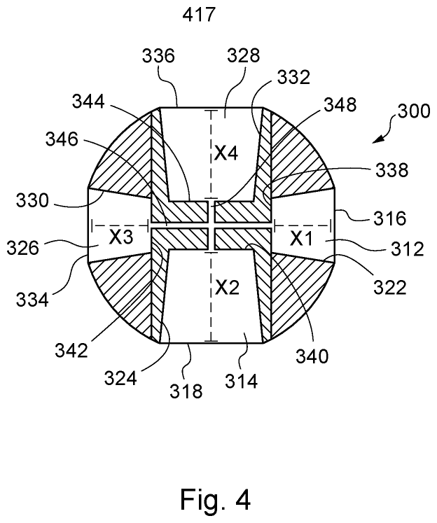

FIG. 4 is a schematic of a third construction of the trial femoral head.

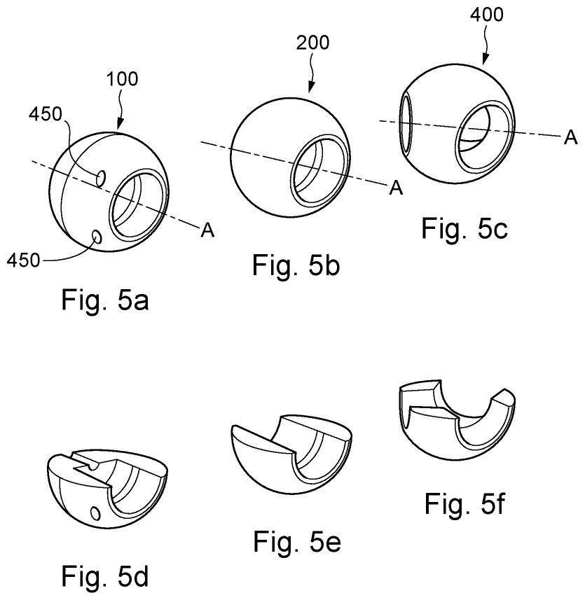

FIG. 5 is a series of perspective and cross-sectional views of a conventional trial femoral head, the first construction of the femoral head shown in FIG. 2 and a fourth construction of the trial femoral head.

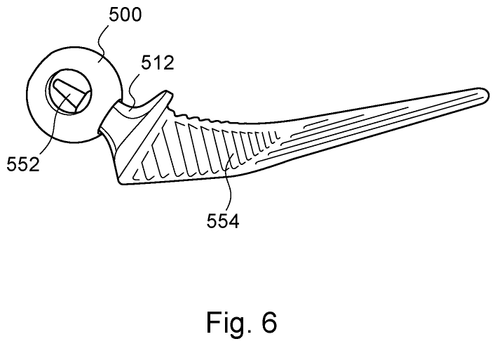

FIG. 6 shows a trial femoral head according to the invention assembled onto a femoral stem component via a first cavity of the head to provide a first neck length.

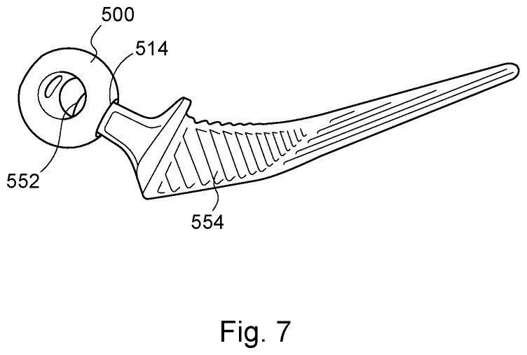

FIG. 7 shows the same trial femoral head of FIG. 6 assembled onto a femoral stem component via a second cavity of the head to provide a second neck length.

Referring now to FIG. 1, there is shown a conventional trial femoral head 10. The trial femoral head has a single cavity 12, shown here in the form of tapered bore. The tapered bore includes a tapered surface 14 which engages with a male tapered connection member at the proximal end of a femoral stem component (not shown). The use of this conventional trial femoral head only allows the surgeon to trial a single neck length. To trial a different neck length, the surgeon must remove this trial femoral head and replace it with a different trial femoral head which has a cavity dimensioned such that when the male tapered connection member is mated with the femoral head it is received at a different depth.

Referring now to FIGS. 2 to 4, there are shown three different constructions of a multi-cavity femoral head according to the invention.

FIG. 2 shows a first construction of the trial femoral head 100 which includes a first cavity 112 and second cavity 114. Each cavity is dimensioned for receipt of a male connection member of a femoral stem component (not shown) at a different depth. Each of the first and second cavities has a male connection member insertion axis, X₁ and X₂, respectively that extends inwardly from an opening 116, 118, located in the generally spherical outer surface 120. It is along the male connection member insertion axis of each cavity that the male connection member of the stem component is inserted and removed when the trial femoral head is connected to and disconnected from the stem component. In the construction shown, axis X₁ and X₂ are coaxial.

The formation of the opening 116, 118 in the outer spherical surface creates an opening that is substantially flat when viewed in a direction substantially orthogonal to the male connection member insertion axis X₁ of the first cavity and the male connection member insertion axis X₂ of the second cavity.

The opening 118 is larger than the opening 116. Each opening can be substantially circular, although other shapes of openings that are configured to receive a complementary shaped male connection member are envisaged.

In the construction shown, each of the first and second cavities has a tapered surface 122, 124, respectively. This tapered surface can engage with the tapered male connection member of the femoral stem component. A Morse taper connection can be formed between the assembled components.

The tapered surface 122 of the first cavity 112 connects with the tapered surface 124 of the second cavity 114. There is not a dividing member (e.g., a wall) between the cavities. An advantage of this inter-connection is that a surgeon can thread a suture through the two cavities, and through adjacent soft tissue. This helps to prevent accidental loss of the trial femoral head within the surgical site.

In use, a surgeon will be able to use the illustrated dual-cavity trial femoral head to trial two different neck lengths. Insertion of the male connection member into the first cavity will provide a first neck length. As a result of the second cavity having a wider opening than the first cavity, the male connection member can be inserted deeper into the second cavity. This will provide a second neck length, which is shorter than the first neck length.

FIG. 3 shows a second construction of the trial femoral head 200. This construction is similar to the dual-cavity construction shown in FIG. 1, apart from the relative positioning of the first and second cavities 212, 214. In this second construction, the male connection member insertion axis, X₁ and X₂, of the first and second cavities are generally orthogonal to each other.

Similar to the first construction of the femoral head, the tapered surface 222 of the first cavity 212 connects with the tapered surface 224 of the second cavity 214. There is not a dividing member (e.g., a wall) between the cavities. An advantage of this inter-connection is that a surgeon can thread a suture through the two cavities, and through adjacent soft tissue. This helps to prevent accidental loss of the trial femoral head within the surgical site.

FIG. 4 shows a third construction of the trial femoral head 300. This construction includes, a first cavity 312, a second cavity 314, a third cavity 326 and a fourth cavity 328.

In the construction shown, each of the first to fourth cavities has a tapered surface 322, 324, 330 and 332, respectively. This tapered surface can engage with the tapered male connection member of the femoral stem component. A Morse taper connection can be formed between the assembled components.

Each of the first to fourth cavities has a male connection member insertion axis, X₁, X₂, X₃ and X₄ that extends inwardly from each opening 316, 318, 334, 336 located in the generally spherical outer surface 320. It is along the male connection member insertion axis of each cavity that the male connection member of the stem component is inserted and removed when the trial femoral head is connected to and disconnected from the stem component. In the construction of the trial femoral head shown in FIG. 4, axis X₁ and X₃ are coaxial, and axis X₂ and X₄ are coaxial. Axis X₁ is generally orthogonal to X₂ and axis X₄. Axis X₃ is generally orthogonal to X₂ and axis X₄.

The formation of each opening in the spherical outer surface of the trial femoral head creates an opening that is substantially flat when viewed in a direction substantially orthogonal to the male connection member insertion axis X₁, X₂, X₃ and X₄ of each of the first to fourth cavities, respectively.

Unlike the trial femoral heads shown in FIGS. 2 and 3 the tapered surfaces of the cavities are not connected. Instead, each cavity includes an end wall 338, 340, 342, 344. Each end wall includes an aperture through which a suture may be threaded. A suture channel 346 extends between the aperture of the first cavity and the aperture of the third cavity. A suture channel 348 extends between the aperture of the second cavity and the aperture of the fourth cavity. To secure the trial head to the surgical site, a surgeon may thread a suture through the first cavity 312, along suture channel 346 and then out through the third cavity 326. Alternatively, a surgeon may thread a suture through the second cavity 314, along suture channel 348 and then out through the fourth cavity 328.

FIG. 5 shows perspective views of (a) the conventional trial femoral head 100 as shown in FIG. 1, (b) the dual-cavity trial femoral head 200 as shown in FIG. 2 and (c) a triple-cavity femoral head 400 according to a further construction of the device. Suture holes 450 are shown in the conventional trial femoral head 100.

FIG. 5 also shows cross-sectional views of (d) head 100, (e) head 200 and (f) head 400 along Plane A. It can be seen that the tapered surfaces of the plurality of cavities in both head 200 and head 400 are connected. This results in an inter-connection between the cavities.

The use of a trial femoral head 500 having four cavities to trial up to four different neck lengths is demonstrated in FIGS. 6 and 7. The trialing of two of the four possible neck lengths achievable by using this trial femoral head are shown.

In FIG. 6 the male connection member 552 of a femoral stem 554 is inserted into a first cavity 512. This cavity has an opening of a first size. The male connection member is inserted into the first cavity at a first depth. In FIG. 7 the same male connection member of the same femoral stem is inserted a second cavity 514. This cavity has an opening that is smaller than the opening of the first cavity. As a result, when the male connection member is inserted into the second cavity it is only insertable to a second depth, this depth being shallower than the first depth. As a result, the use of the second cavity provides an assembly between the trial femoral head and the stem component that has a longer neck length than the assembly formed when the first cavity is used. This variability is provided by a single trial femoral head.

1. A trial femoral head for use in a trial fitting of a hip prosthesis, the head having an outer surface and a plurality of cavities configured to mate with a male connection member of a femoral stem component, each cavity having an opening located in the outer surface of the head, each opening being of a different size such that when the male connection member of the femoral stem component is mated with a first cavity it is received at a first depth, and when it is mated with a second cavity it is received at a second depth.

2. The trial femoral head of claim 1, wherein each cavity comprises a tapered surface to engage with a tapered male connection member.

3. The trial femoral head of claim 2, wherein a taper angle of the tapered surface of each cavity is the same.

4. The trial femoral head of claim 1, wherein each cavity has a male connection member insertion axis extending inwardly from the opening and wherein the male connection member insertion axis of the first cavity is coaxial with the male connection member insertion axis of the second cavity.

5. The trial femoral head of claim 1, wherein each cavity has a male connection member insertion axis and wherein the male connection member insertion axis of the first cavity is oriented at a non-zero angle relative to the male connection member insertion axis of the second cavity.

6. The trial femoral head of claim 5, wherein the male connection member insertion axis of the first cavity is orthogonal to the male connection member insertion axis of the second cavity.

7. The trial femoral head of claim 2, wherein the tapered surface of the first cavity connects with the tapered surface of the second cavity.

8. The trial femoral head of claim 4, wherein the head further includes a third cavity which has a male connection member insertion axis that is oriented at a non-zero angle relative to the male connection member insertion axis of at least one of the first and second cavities.

9. The trial femoral head of claim 8, wherein the male connection member insertion axis of the third cavity is orthogonal to the male connection member insertion axis of at least one of the first and second cavities.

10. The trial femoral head of claim 8, wherein the third cavity comprises a tapered surface to engage with a tapered male connection member, and wherein the said tapered surface connects with the tapered surface at least one of the first and second cavities.

11. The trial femoral head of claim 8, wherein the head further includes a fourth cavity which has a male connection member insertion axis that is oriented at a non-zero angle relative to the male connection member insertion axis of at least one of the first and second cavities.

12. The trial femoral head of claim 11, wherein the male connection member insertion axis of the fourth cavity is orthogonal to the male connection member insertion axis of at least one of the first and second cavities.

13. The trial femoral head of claim 11, wherein the male connection member insertion axis of the fourth cavity is co-axial with the male connection member insertion axis of the third cavity.

14. The trial femoral head of claim 11, wherein the fourth cavity comprises a tapered surface to engage with a tapered male connection member, and wherein the said tapered surface connects with the tapered surface at least one of the first, second and third cavities.

15. The trial femoral head of any preceding claim, wherein the outer surface is generally spherical.

16. The trial femoral head of claim 4, wherein each opening is substantially flat when viewed in a direction substantially orthogonal to the male connection member insertion axis.

17. A kit comprising first and second trial femoral heads for use in a trial fitting of a hip prosthesis, each head having an outer surface and a plurality of cavities configured to mate with a male connection member of a femoral stem component, each cavity having an opening located in the outer surface of each head, each opening being of a different size such that when the male connection member of the stem component is mated with a first cavity it is received at a first depth, and when it is mated with a second cavity it is received second depth, and wherein the outer surface of the first trial femoral head has a diameter that is different from a diameter of the outer surface of the second trial femoral head.

18-22. (canceled)
